(19) United States
(12) Patent Application Publication
(10) Pub. No.: US 2004/0022922 A1
(43) Pub. Date: Feb. 5, 2004

(54) Infant formula supplemented with phospholipids

(76) Inventor: David Rutenberg, Haifa (IL)

Correspondence Address: Mark Friedman, Bill Polkinghorn, Discovery Dispatch, 9003 Florin Way, Upper Marlboro, MD, 20772, US

(21) Appl. No.: 10/275,748

(22) PCT Filed:
(86) PCT No.: PCT/IL02/00674

(30) Foreign Application Priority Data
Jun. 16, 2002 (IL) 150240

(51) Int. Cl. A23D 7/00

(52) U.S. Cl. 426/601

(57) ABSTRACT

The present invention relates to pharmaceutical or nutritional or food composition for feeding infants which is more similar to human milk in a powder or fluid form, having an effect of addressing the shortcomings of the presently known infant formulas by the inclusion of a phospholipid supplement which contains at least 1% (w/w) phosphatidyserine out of the total phospholipid content of the composition wherein the phosphatidylserine has a structural fatty acid chain derived from at least one raw material lecithin selected from the group consisting of soy bean lecithin, rapeseed lecithin, or egg yolk lecithin, and which is produced by reaction pith phospholipase-D.

### FIELD AND BACKGROUND OF THE INVENTION

The present invention relates to an infant formula supplemented with glycerophospholipids (hereinafter phospholipids) and more particularly, to a formula supplemented with phosphatidylserine.

Breastfeeding, the natural feeding mode, has multiple beneficial effects on the infant and is known to be the most suitable diet for infant's nutritional requirements.

Jensen R G. (Textbook of Gastroenterology and Nutrition in Infancy, Second Edition, edited by E. Lebenthal. Raven Press, Ltd., pp. 157-208, New York 1989) reviewed measurements of the total lipid content of human milk and reports that phospholipids are found in the human milk lipids at levels of about 20 to 40 mg/dl.

G. Harzer et al. reports that the phospholipid composition of human milk lipids (weight % of total phospholipids) in day 36 postpartum is phosphatidylcholine (PC) 24.9%, phosphatidylethanolamine (PE) 27.7%, phosphatidylserine (PS) 9.3%, phosphatidylinositol (PI) 5.4%, sphingomyelin 32.4% (G. Harzer et al., Am J Clin Nutr., Vol. 37, pp. 612-621, 1983).

Phospholipids are essential components of growing tissue in particular nerve cells. In the latter, lack of phospholipids can cause serious impairment in development and problems in overt functions. The source of phospholipids in the buildup of neuronal tissue, originates in part from intracellular synthesis and in part from the diet. Therefore dietary supplementation of phospholipids in lactating infants could be critical for proper development (For comprehensive reviews, see: J. N. Hawthorne & G. B. Ansell, Phospholipids 1982, Elsevier, Amsterdam, and M. Shinitzky, “Membrane Fluidity and Cellular Function”, in Physiology of Membrane Fluidity, Shinitzky M. Ed. Vol. 1, pp. 1-53, 1984, CRC Press, Boca Raton, Fla.).

In the past, only pig or bovine phosphatidylserine was available in the market. However, due to the “mad cow disease” this source is generally unacceptable for usage with infant formulations today. In recent years a reliable and healthy source of phosphatidylserine, which is produced by reaction of vegetal lecithin with phospholipase-D is available in the market. A specific process of enzymatic conversion is known and is described in, for example, the article by Eibl A. and Kovatchev S. (Eibl A. and Kovatchev S. “Methods in Enzymology” Vol. 72, pp.: 632-639, 1981).

### REFERENCES

Jensen R G. “Textbook of Gastroenterology and Nutrition in Infancy”, Second Edition, edited by E. Lebenthal. Raven Press, Ltd., pp. 157-208, New York 1989.

G. Harzer et al., “Changing patterns of human milk lipids in the course of lactation and during the day,”, Am J Clin Nutr., Vol. 37, pp. 612-621.

J. N. Hawthorne & G. B. Ansell, Phospholipids 1982, Elsevier, Amsterdam.

Shinitzky M. Ed “Membrane Fluidity and Cellular Function in Physiology of Membrane Fluidity”, Vol. 1, pp. 1-53, 1984, CRC Press, Boca Raton, Fla.

Eibl A. and Kovatchev S. “Preparation of phospholipids analogs by phospholipase-D” Methods in Enzymology” Vol. 72, pages: 632-639, 1981.

### SUMMARY OF THE INVENTION

It is an object of the present invention to provide an infant formula which is more similar to human milk in a powder or solution form, having an effect of addresseing the shortcomings of the presently known infant formulas by the inclusion of a phospholipids supplement which contains phosphatidylserine as one of the effective ingredients.

The infant formula of the present invention contains phosphatidylserine or the salt thereof as the effective ingredient, wherein the phosphatidylserine has a structural fatty acid chain derived from at least one raw material lecithin selected from the group consisting of soy bean lecithin, rapeseed lecithin, or egg yolk lecithin.

According to further features in preferred embodiments of the invention described below, the phospholipids include phosphatidylserine or the salt thereof as the effective ingredient, wherein the phosphatidylserine derived from at least one raw material lecithin selected from the group consisting of soy bean lecithin, rapeseed lecithin, or egg yolk lecithin, and which is produced by reaction with phospholipase-D.

The aforementioned phosphatidylserine as the effective ingredient in accordance faith the present invention is produced by the enzymatic reaction with phospholipase-D using as the substrate soy bean lecithin, rapeseed lecithin or ego yolk lecithin. The process will now be illustrated. A raw material lecithin (namely, phosphatidylcholine) selected from soy bean lecithin, rapeseed lecithin or egg yolk lecithin is subjected to the process of transphosphatidylation with phospholipase-D in the presence of L-serine or D-serine or DL-serine and water, thereby substituting the choline group with the serine group through the hydroxyl group, to produce the rearranged phosphatidylserine.

Any commercially available soy bean lecithin, rapeseed lecithin or egg yolk lecithin may be used, with no limitation, as the raw material. As phospholipase-D for use in the process of enzymatic conversion, use may be made of for example those from cabbage and actinomyces, if they have an activity on lecithin or hydrogenated lecithin or lysolecithin in the presence of L-serine or D-serine or DL-serine and water to produce phosphatidylserine.

A specific process of enzymatic conversion is known and described in for example the article by Eibl A. and Kovatchev S. “Preparation of phospholipids analogs by phospholipase-D.” (“Methods in Enzymology” Vol. 72, pp.: 632-639, 1981), so no detailed explanation is described herein.

According to another aspect of the present invention there is provided a method of feeding an infant comprising the steps of mixing an infant formula powder in water for obtaining a fluid including the nutritional components and the phospholipids supplement and feeding the infant with the fluid containing nutritional components and a phospholipid supplement wherein the phospholipids include phosphatidylserine or the salt thereof as the effective ingredient.

According to still further features in the described preferred embodiments at least some of the nutritional components are derived from milk or soy.

The present invention successfully addresses the shortcomings of the presently known configurations by providing an infant formula which is more similar to human milk, adds protection from serious impairment in development and problems in overt functions and improve the development and maturation of tissue in nerve cells.

### DESCRIPTION OF THE PREFERRED EMBODIMENTS

The present invention is of an infant formula supplemented with phospholipids, particularly which includes phosphatidylserine, which can be used to feed infants. Specifically, the present invention can be used to protect infants of syndromes associated with feed devoid of phospholipids fed to them in the first year of their lives. The present invention renders infant formulas more similar to human milk.

Thus, in accordance with one aspect of the present invention there is provided an infant formula in a powder or fluid form which formula includes nutritional components and a phospholipid supplement, particularly which includes phosphatidylserine.

In accordance with another aspect of the present invention there is provided a method of feeding an infant. The method is effected by executing the following steps: First an infant formula powder containing nutritional components and a phospholipid supplement is mixed in water for obtaining a mixture including the nutritional components and the phospholipid supplement. Second, the mixture is fed to the infant.

As exemplified in the Examples section below, the nutritional components may include milk or soy derived nutritional components. They may additionally include one or more of the following ingredients: lactose, vegetable oils, skimmed milk powder, whey protein concentrate, Sodium, Calcium, Phosphorus, Potassium, Chloride, Iron, Magnesium, Taurine, Vitamins, Glucose syrup, soy protein isolate, Sucrose, Maltodextrine, Methionine, Taurine, Carnitine, and trace elements.

Further exemplified in the Examples section below, is the preparation of phosphatidylserine and the phospholipid complex thereof.

### EXAMPLE 1-1

Tables 1 and 2 below provide exemplary compositions of dry and fluid milk based infant formulas and dry and fluid soy based infant formulas according to the present invention.

Infant Formula I (Milk-Based)

The formula comprises the following ingredients: lactose, vegetable oils, skimmed milk powder, whey protein concentrate, Sodium, Calcium, Phosphorus, Potassium, Chloride, Iron, Magnesium, Taurine, Vitamins and phospholipids.

TABLE 1

| | | Powder | fluid |
|---|---|---|---|
| | Unit | 100 g | 100 ml |
| General Comp. | | | |
| Protein | gram | 11.1 | 1.5 |
| Fat | gram | 25.9 | 3.5 |
| Lactose | gram | 55.5 | 7.5 |
| Water | gram | 2.5 | — |
| Ash | gram | 2.06 | 0.27 |
| Vitamins | | | |
| Vitamin A | I.U. | 1500 | 200 |
| Vitamin D | I.U. | 300 | 40 |
| Vitamin E | mg | 6 | 0.81 |
| Vitamin K | .mu · g | 15 | 2.01 |
| Vitamin B1 | .mu · g | 350 | 47.03 |
| Vitamin B2 | .mu · g | 450 | 60 |
| Vitamin B6 | .mu · g | 222 | 30 |
| Vitamin B12 | .mu · g | 0.66 | 0.09 |
| Niacin | mg | 2 | 0.27 |
| Folic Acid | .mu · g | 45 | 6 |
| Calcium | mg | 4.44 | 0.06 |
| Pantothenate | | | |
| Biotin | .mu · g | 11 | 1.5 |
| Vitamin C | mg | 45 | 6.08 |
| Minerals | | | |
| Calcium | mg | 326 | 44 |
| Phosphorus | mg | 219 | 29.6 |
| Magnesium | mg | 37 | 5 |
| Iron | mg | 7.4 | 1 |
| Sodium | mg | 120.7 | 16.3 |
| Potassium | mg | 373 | 50 |
| Ca/p ratio | — | 1.49 | 1.49 |
| Amino Acid Profile | | | |
| Alanine | mg | 522 | 69.6 |
| Arginine | mg | 368 | 49.1 |
| Asparatic Acid | mg | 11.10 | 1.5 |
| Cystine | mg | 191 | 25.5 |
| Glutamic Acid | mg | 1423 | 189.7 |
| Glycine | mg | 244 | 32.5 |
| Histidine | mg | 262 | 34.9 |
| Isoleucine | mg | 761 | 101.5 |
| Leucine | mg | 12.20 | 1.62 |
| Lysine | mg | 10.00 | 1.3 |
| Methionine | mg | 270 | 36 |
| Phenylalanine | mg | 461 | 62.3 |
| Proline | mg | 962 | 128.3 |
| Serine | mg | 681 | 90.8 |
| Taurine | mg | 37 | 4.9 |
| Threonine | mg | 686 | 91.5 |
| Tryptophan | mg | 180 | 24 |
| Tyrosine | mg | 463 | 61.7 |
| Valine | mg | 775 | 103.3 |
| Fatty Acid Profile | | | |
| Caprylic (C8) | % from fat | 2.6 | 2.6 |
| Capric (C10) | % | 2.1 | 2.1 |
| Lauric (C12) | % | 17.5 | 17.5 |
| Meristic (C14) | % | 6.7 | 6.7 |
| Palmitic (C16) | % | 11.2 | 11.2 |
| Stearic (C18) | % | 11.8 | 11.8 |
| Oleic (C18:1) | % | 37.0 | 37.0 |
| Linoleic (C18:2) | % | 10.0 | 10.0 |
| Linolenic (C18:3) | % | 1.2 | 1.2 |
| Phospholipid supplement | | | |
| which is added to the | | | |
| aforementioned 100 g. | | | |
| of powder or fluid): | | | |
| Phosphatidylcholine | mg | 250.0 | 250.0 |
| Phosphatidylethanolamine | mg | 250.0 | 250.0 |
| Phosphatidylserine | mg | 100.0 | 100.0 |
| Phosphatidylinositol | mg | 50.0 | 50.0 |
| Phosphatidic Acid | mg | 50.0 | 50.0 |

Infant Formula II (Soy-Based)

The formula comprises the following ingredients: Glucose syrup, vegetable oils, soy protein isolate, Sucrose Maltodextrine, Sodium, Calcium, Phosphorus, Potassium, chloride, Iron, Magnesium, Vitamins, Methionine, Taurine, Camitine, trace elements and phospholipids.

TABLE 2

| | | Powder | fluid |
|---|---|---|---|
| | Unit | 100 g | 100 ml |
| General Comp. | | | |
| Protein | gram | 15 | 1.98 |
| Fat | gram | 27.54 | 3.64 |
| Carbohydrate | gram | 51.5 | 6.8 |
| Linoleic Acid | gram | 4.5 | 0.6 |
| Vitamins | | | |
| Vitamin A | I.U. | 1500 | 198 |
| Vitamin D | I.U. | 300 | 39.7 |
| Vitamin E | I.U. | 10 | 1.32 |
| Vitamin C | mg | 65 | 8.6 |
| Vitamin K | .mu · g | 77 | 10.2 |
| Vitamin B1 | .mu · g | 345 | 45.6 |
| Vitamin B2 | .mu · g | 445 | 58.9 |
| Vitamin B6 | .mu · g | 327 | 43.3 |
| Vitamin B12 | .mu · g | 1.5 | 0.2 |
| Niacin | mg | 7 | 0.93 |
| Folic Acid | .mu · g | 76 | |
| Pantothenic Acid | .mu · g | 4.5 | 0.6 |
| Biotin | .mu · g | 25 | 3.3 |
| Choline | mg | 58 | 7.7 |
| Minerals | | | |
| Calcium | mg | 500 | 66.2 |
| Phosphorus | mg | 300 | 39.7 |
| Magnesium | mg | 45 | 6 |
| Iron | mg | 9.2 | 1.2 |
| Zinc | mg | 4 | 0.53 |
| Manganese | .mu · g | 150 | 19.8 |
| Copper | .mu · g | 400 | 53 |
| Iodine | .mu · g | 77 | 10.2 |
| Sodium | mg | 200 | 26.5 |
| Potassium | mg | 546 | 72.2 |
| Chloride | mg | 400 | 53 |
| Inositol | mg | 25 | 3.3 |
| Carnitine | mg | 10 | 1.3 |
| Ca/P ratio | | 1.67 | 1.67 |
| Amino Acid Profile | | | |
| Alanine | mg | 640 | 85.3 |
| Arginine | mg | 497 | 6.5 |
| Aspartic Acid | mg | 1385 | 184.7 |
| Cystine | mg | 242 | 32.3 |
| Glutamic Acid | mg | 3065 | 408.7 |
| Glycine | mg | 300 | 40 |
| Histidine | mg | 382 | 50.9 |
| Isoleucine | mg | 893 | 119.1 |
| Leucine | mg | 1600 | 213.3 |
| Lysine | mg | 1360 | 181.3 |
| Methionine | mg | 406 | 54.1 |
| Phenylalanine | mg | 650 | 86.7 |
| Proline | mg | 1113 | 148.4 |
| Serine | mg | 737 | 98.3 |
| Taurine | mg | 51 | 6.8 |
| Threonine | mg | 460 | 61.3 |
| Tyrosine | mg | 621 | 82.8 |
| Valine | mg | 947 | 126.3 |
| Fatty Acid Profile | | | |
| Caprylic (C8) | % from fat | 2.6 | 2.6 |
| Capric (C10) | % | 2.1 | 2.1 |
| Lauric (C12) | % | 17.5 | 17.5 |
| Meristic (C14) | % | 6.7 | 6.7 |
| Palmitic (C16) | % | 11.2 | 11.2 |
| Stearic (C18) | % | 11.8 | 11.8 |
| Oleic (C18:1) | % | 37.0 | 37.0 |
| Linoleic (C18:2) | % | 10.0 | 10.0 |
| Linolenic (C18:3) | % | 1.2 | 1.2 |
| Phospholipid supplement | | | |
| which is added to the | | | |
| aforementioned 100 g. | | | |
| of powder or fluid): | | | |
| Phosphatidylcholine | mg | 250.0 | 250.0 |
| Phosphatidylethanolamine | mg | 250.0 | 250.0 |
| Phosphatidylserine | mg | 100.0 | 100.0 |
| Phosphatidylinositol | mg | 50.0 | 50.0 |
| Phosphatidic Acid | mg | 50.0 | 50.0 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### EXAMPLE 2-1

Soybean lecithin (50 g; Epikuron 135 as the product name; Lucas Meyer GmbH, Germany) and soybean oil (10 g) were placed in a 300-ml vial, followed by addition of ethyl acetate (50 ml) for solubilization. Adding a solution (20 ml) of 0.30 g/ml L-serine dissolved in 0.1M sodium phosphate buffer, pH 7.0 to the resulting solution for thorough blending, a solution of 500 U/ml phospholipase-D from cabbage was added to the mixture solution for reaction at 25. degrees C. for 5 hours under stirring with a stirrer.

For inactivation of the enzyme in the reaction solution, the vial containing the reaction solution was immersed in hot water. Subsequently, the reaction solution was cooled in ice and treated with chloroform. The chloroform layer was collected and dried under reduced pressure.

### EXAMPLE 2-2

Using egg yolk lecithin (DS-PL95E as the product name; manufactured by Doosan Corp. Venture BG Biotech BU. Korea) as the substrate, rearranged phosphatidylserine was produced by the same method as in Example 1-1.

### EXAMPLE 3

Phosphatidylserine was prepared by Lipogen Products (9000) Ltd. via the process of enzymatic reaction from a substrate soybean lecithin according to Example 2-1. 50 grams of this preparation were added to 50 grams of soy lecithin containing high percentage of phosphatidylcholine (50 g; Epikuron 145V as the product name; Lucas Meyer GmbH, Germany). The mixture was vigorously stirred to produce a homogeneous phospholipids complex.

What is claimed is:

1. A pharmaceutical or nutritional or food composition for feeding infants, which is more similar to human milk in a powder or fluid form having an effect of addresseing the shortcomings of the presently known infant formulas by the inclusion of a phospholipid supplement which contains phosphatidylserine as one of the effective ingredients.

2. The composition of claim 1 comprising of a phospholipid complex including phosphatidylserine or salts thereof as one of the effective ingredient, comprising at least 1% (w/w) phosphatidyserine out of the total phospholipid content of the composition, wherein the phosphatidylserine has a structural fatty acid chain derived from at least one raw material lecithin selected from the group consisting of soy bean lecithin, rapeseed lecithin, or ego yolk lecithin, and which is produced by reaction with phospholipase-D.

3. The composition of claim 1, comprising of a phospholipid complex including phosphatidylserine or salts thereof as one of the effective ingredient, comprising at least 1% (w/w) phosphatidyserine out of the total phospholipid content of the composition, wherein the phosphatidyserine has a structural fatty acid chain derived from at least one raw material lecithin selected from the group consisting of soy bean lecithin, rapeseed lecithin, or egg yolk lecithin and which is produced by reaction with phospholipase-D and wherein the structural fatty acid chain is a hydrogenated saturated fatty acid chain.

4. The composition of claim 1 which also contains milk or soy derived nutritional ingredients.

5. The composition of claim 1 which may also contain one or more of the following ingredients: lactose, vegetable oils, skimmed milk powder, whey protein concentrate, Sodium, Calcium, Phosphorus, Potassium, Chloride, Iron, Magnesium, Taurine, Vitamins, Glucose syrup, soy protein isolate, Sucrose, Maltodextrine, Methionine, Taurine, Carnitine, and trace elements.

6. The composition of claim 1 which also contains pharmaceutical or food excipient.

7. The composition of claim 1 which is mixed in water for obtaining a fluid including the nutritional components and the phospholipid supplement and which is fed to the infant.
